(19) United States
(12) Patent Application Publication
(10) Pub. No.: US 2023/0285699 A1
(43) Pub. Date: Sep. 14, 2023

(54) MANUAL VENTILATORS AND METHODS FOR MAKING VENTILATORS

(71) Applicant: United States of America as represented by the Administrator of NASA, Washington, DC (US)

(72) Inventors: James P. Locke, Houston, TX (US); Holly P. Newton, Houston, TX (US); Mark E. Schaefbauer, Houston, TX (US); Molly E. Harwood, Houston, TX (US)

(21) Appl. No.: 17/999,875

(22) PCT Filed: Jun. 2, 2021
(86) PCT No.: PCT/US2021/035558

§ 371 Date: Nov. 25, 2022

Related U.S. Application Data
(60) Provisional application No. 63/034,485, filed on Jun. 4, 2020.

(51) Int. Cl. A61M 16/00; A61M 16/20

(52) U.S. Cl. CPC A61M 16/0075; A61M 16/208; A61M 2205/071; A61M 2205/8281

(57) ABSTRACT

Presented are manual ventilator systems, methods for making/using such ventilator systems, and clamshell, accordion-style ventilators operable by a lone operator. A ventilator device includes first and second panels pivotably attached together in a clamshell configuration. Each panel has an inlet port, an outlet port, and an internal channel that fluidly connects the inlet and outlet ports. A one-way valve is fluidly connected to the first panel’s outlet port and restricts airflow therethrough in one direction. Another one-way valve is fluidly connected to the second panel’s inlet port and restricts airflow therethrough in an opposite direction. A concertinaed bellows is fluidly connected to the first panel’s outlet port and the second panel’s inlet port. The bellows is sandwiched between and attached to the two panels such that pivoting the panels away from each other expands and fills the bellows whereas pivoting the panels towards each other compresses and evacuates the bellows.

### CROSS-REFERENCE TO RELATED PATENT APPLICATION

This patent application claims the benefit of priority to U.S. Provisional Pat. Application No. 63/034,485, which was filed on Jun. 4, 2020, and is incorporated herein by reference in its entirety and for all purposes.

### ORIGIN OF THE INVENTION

The invention described herein was made by employees of the United States Government and may be manufactured and used by or for the Government of the United States of America for governmental purposes without the payment of any royalties thereon or therefor. The invention described herein was also made in the performance of work under a NASA contract and is subject to the provisions of the National Aeronautics and Space Act of 1958, Public Law 85-568, now codified at 51 USC § 20135.

### TECHNICAL FIELD

The present disclosure relates generally to assisted ventilation devices. More specifically, aspects of this disclosure relate to manually powered ventilators and methods for making and methods for using manual ventilators.

### BACKGROUND

Respiratory ventilation or resuscitation is typically performed on an individual that is unable to breathe normally without assistance, for example, due to injury or illness. There are three primary options for assisted respiratory ventilation/resuscitation: (1) ventilator machines, (2) manual ventilators/resuscitators, and (3) direct-application manual ventilation (e.g., cardiopulmonary resuscitation (CPR)). Modern-day electrical or gas-powered ventilator machines are microprocessor-controller devices typically found in advanced health care settings. While quite effective, most ventilator machines now require complex electronics in their build for operation, trained and skilled personnel during operation, and regular maintenance.

Existing manual resuscitators, such as a hand-operated bag valve mask (BVM), are designed for use in short-duration resuscitation scenarios and are ill-suited for continuous, prolonged usage. The small muscles of the hand and wrist are used to squeeze the BVM bag, and those muscles quickly fatigue with repetitive use. In particular, the repetitive contraction and extension of the small muscles in a person’s hand, wrist, and forearm to compress and expand the respirator bag repeatedly for lengthy periods may cause fatigue that leads to slowed and erratic pumping. A single user can therefore only operate the device effectively for a few minutes at a time. In order to provide respiratory support for long periods of time with a BVM, a number of operators need to be available to trade off device operation. Oftentimes, however, an inadequate number of operators are available to provision long-term BVM use, making BVMs impractical for many scenarios. Moreover, some BVM-type ventilators have complex designs requiring a large number of interconnected parts, increased manufacturing time and costs, and complex instructions for operating the device. Device cost and complexity limits the availability of such BVM-type ventilators for use by inexperienced operators and in resource-poor demographics.

### SUMMARY

Presented herein are manual ventilator systems, methods for making and methods for using manual ventilator systems, and clamshell, accordion-style ventilator devices operable by a lone operator. As a non-limiting example, disclosed manual ventilators employ a pleated bellows that is sandwiched between and fluidly sealed to a pair of juxtaposed (intake and exhaust) panels. Longitudinal ends of these elongated panels are joined via a pivot hinge in a clamshell form factor. Proximal ends of the panels may be fashioned with handles to improve operator gripping for hand pumping of the ventilator. Biasing members, such as helical springs or torsion springs, bias the panels apart to facilitate expansion and filling of the bellows. When the two panels are pivoted away from each other, ambient air and/or tank-fed oxygen is drawn in through an internal fluid channel of the intake panel, an intake flutter/check valve mounted to the intake panel opens, and an exhaust flutter/check valve mounted to the exhaust panel closes, filling the bellows with air/oxygen. Conversely, when the two panels are pressed together, the intake flutter/check valve closes, the exhaust flutter/check valve opens, and air/oxygen is expelled out of the bellows through an internal fluid channel of the exhaust panel.

Manual ventilators eliminate the need for electrical power and complex electronics to provide respiratory ventilation or resuscitation. Use of an accordion-like pumping operation is intuitive and simple, which minimizes any requisite operator training. In addition, the operator may be provided with tactile sensory feedback, e.g., to feel physically the volume and rate of air delivered to a patient, which helps to ensure consistent and safe ventilation. Optional air pressure sensors and air flow sensors may be placed in-line with the device’s intake/exhaust ports to assist the operator further. An optional dual-ported attachment to the air inlet port allows for supplemental oxygen to be delivered, for example, from an oxygen tank and/or a reservoir bag. The manual ventilator may be strapped to the operator’s leg (e.g., for ease of transport and single-hand operation), placed on a table (for single-hand operation), or held by the operator (e.g., for dual-hand operation). For purposes of this disclosure, the terms “manual” and “human-powered” may be used interchangeably and synonymously in a non-limiting fashion to reference devices physically operated by a human that do not require electrical power or electrical components to function properly.

Disclosed ventilators may be used for any logically relevant application, including for sick, injured, or sedated individuals, for infants, children, or adults, for private or professional use, for intubation or for mask designs, etc. The ventilator device may be manufactured from inexpensive materials and, if desired, disposed of after a single use or, alternatively, may be sterilized between uses. For modular designs, only the air modules and bellows are disposed of after each use; the intake and exhaust panels, clamshell pivot joints, end caps, springs, etc., can be reused. Moreover, disclosed lightweight and compact manual ventilator designs help to simplify operation, shipping, storage, and portability of the device.

Attendant benefits for at least some of the concepts disclosed herein may include manual ventilator designs that provide the operator with tactile feedback to deliver optimal breaths to the patient. Simplified and intuitive control of the device allows for minimally trained operators to assist with respiration for longer durations compared to existing manual ventilators. Disclosed devices also use larger muscles (e.g., shoulder, chest, back, legs, arms, etc.) rather than small muscles (anterior, posterior, and digitorum hand muscles) to minimize operator fatigue. Disclosed designs herein may allow a single person to operate the manual ventilator device for more than an hour at a time. Simplified designs reduce part count and complexity, which in turn diminishes manufacturing time and costs as well as repair and replacement costs. Reducing system cost and complexity makes manual ventilators as described herein accessible to inexperienced operators and to resource-poor demographics.

Aspects of this disclosure are directed to mechanical ventilator systems and devices for assisted respiratory ventilation/resuscitation. In an example, a ventilator device includes two (first and second) panels that are pivotably attached to each other, e.g., in a clamshell configuration. Each panel defines a respective inlet port, a respective outlet port, and a respective internal channel that fluidly connects the inlet and outlet ports. A (first) one-way airflow valve fluidly connects to the first panel’s outlet port, restricting airflow therethrough in an outward (first) direction. Another (second) one-way valve fluidly connects to the second panel’s inlet port, restricting airflow therethrough in an inward (second) direction, opposite that of the outward (first) direction. A concertinaed bellows is fluidly connected to the first panel’s outlet port and the second panel’s inlet port, e.g., seated against each panel’s internal face and circumscribing each port’s opening. In addition, the bellows is sandwiched between and attached, e.g., via clamps, straps, screws, etc., to the two panels such that pivoting the panels away from each other expands (and fills) the bellows, whereas pivoting the two panels towards each other compresses (and evacuates) the bellows.

Other aspects of this disclosure are directed to methods for manufacturing and methods operating manual ventilators for assisted respiratory ventilation/resuscitation. In an example, a method is presented for assembling a ventilator device. This representative method includes, in any logical order and in any logical combination with any of the above and below disclosed options and features: receiving a first panel defining a first inlet port, a first outlet port, and a first internal channel fluidly connecting the first inlet and outlet ports; fluidly connecting a first one-way valve to the first outlet port, the first one-way valve restricting airflow through the first outlet port in a first direction; pivotably attaching a second panel to the first panel in a clamshell configuration, the second panel defining a second inlet port, a second outlet port, and a second internal channel fluidly connecting the second inlet and outlet ports; fluidly connecting a second one-way valve to the second inlet port, the second one-way valve restricting airflow through the second inlet port in a second direction opposite the first direction; fluidly connecting a bellows to the first outlet port and the second inlet port; and attaching the bellows to the first and second panels with the bellows interposed between the first and second panels such that pivoting the first and second panels away from each other expands the bellows, drawing air through the first panel and into the bellows, and pivoting the first and second panels towards each other compresses the bellows, expelling air from the bellows and out through the second panel.

For any of the disclosed systems, methods, and devices, each panel may include an elongated and substantially flat panel body with a longitudinal end that defines therethrough the inlet (or outlet) port and a major face that defines therethrough the outlet (or inlet) port. As yet a further option, each panel may include an annular or polyhedral mounting interface that projects inwardly from the major face of the panel body. The first panel’s mounting interface surrounds the first outlet port, whereas the second panel’s mounting interface surrounds the second inlet port. In this instance, each opposing end of the bellows is mounted to a respective one of the mounting interfaces. For ease of design and simplicity of manufacture, the panels may be substantially structurally identical.

For any of the disclosed devices, systems, and methods, a pivot hinge may pivotably attach a longitudinal end of the first panel to a longitudinal end of the second panel. The pivot hinge may be a barrel-type hinge with a respective barrel sleeve and a respective barrel cylinder attached to the longitudinal end of each panel. In this instance, the first panel’s barrel cylinder slides into and rotates inside of the second panel’s barrel sleeve, whereas the second panel’s barrel cylinder slides into and rotates inside of the first panel’s barrel sleeve. As yet a further option, each panel may be integrally formed with its respective barrel cylinder and barrel sleeve. Conversely, the barrel cylinder and barrel sleeve may be fabricated as distinct joints that are subsequently mounted to opposing sides of the panel body. In either instance, each panel’s internal channel may extend through the barrel cylinder, e.g., to fluidly connect to a hose or reservoir bag via an adapter end cap.

For any of the disclosed devices, systems, and methods, the one-way valves may be flapper-type valves with flexible flapper discs. The flapper disk of the first panel’s flapper valve seats against an exterior surface of the first panel body, e.g., inside the annular mounting interface. Conversely, the flapper disk of the second panel’s flapper valve seats against an interior surface of the second panel body, e.g., inside the internal channel. As yet a further option, each of the panels may include a hand-operated handle, such as a U-shaped, through-hole handle. An optional biasing member may be employed to bias the two panels away from each other. The biasing member may include one or more, helical springs, torsion springs, etc.

The above summary does not represent every embodiment or every aspect of this disclosure. Rather, the above features and advantages, and other features and attendant advantages of this disclosure, will be readily apparent from the following detailed description of illustrative examples and modes for carrying out the present features when taken in connection with the accompanying drawings and the appended claims. Moreover, this disclosure expressly includes any and all combinations and subcombinations of the elements and features presented above and below.

### BRIEF DESCRIPTION OF THE DRAWINGS

FIG. 1 is an elevated, perspective-view illustration of a representative manual ventilator device according to aspects of the disclosed concepts.

FIG. 2 is an exploded, perspective-view illustration of select parts of the representative manual ventilator device of FIG. 1.

FIG. 3 is an elevated, perspective-view illustration of another representative manual ventilator device according to aspects of the disclosed concepts.

FIG. 4 is an exploded, perspective-view illustration of select parts of the representative manual ventilator device of FIG. 3.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover all modifications, equivalents, combinations, subcombinations, permutations, groupings, and alternatives falling within the scope of this disclosure as encompassed, for instance, by the appended claims.

### DETAILED DESCRIPTION

This disclosure is susceptible of embodiment in many different forms. Representative examples of the disclosure are shown in the drawings and herein described in detail with the understanding that these embodiments are provided as an exemplification of the disclosed principles, not limitations of the broad aspects of the disclosure. To that end, elements and limitations that are presented in the Abstract, Technical Field, Background, Summary, Description of the Drawings, and Detailed Description sections, but not explicitly set forth in the claims, should not be incorporated into the claims, singly or collectively, by implication, inference, or otherwise. For purposes of the present detailed description, unless specifically disclaimed: the singular includes the plural and vice versa; the words “and” and “or” shall be both conjunctive and disjunctive; the words “any” and “all” shall both mean “any and all”; and the words “including,” “containing,” “comprising,” “having,” permutations thereof, and alike terms, shall each mean “including without limitation.” Moreover, words of approximation, such as “about,” “almost,” “substantially,” “generally,” “approximately,” and the like, may each be used herein in the sense of “at, near, or nearly at,” or “within 0-5% of,” or “within acceptable manufacturing tolerances,” or any logical combination thereof.

Discussed below are manual ventilator devices for providing assisted respiratory ventilation or resuscitation. Generally speaking, the illustrated ventilator designs use a concertinaed bellows that is sandwiched between two pivot-hinged panels that are operated with an accordion-like pumping motion to deliver air to a recipient. Tactile sensory feedback may be provided to the operator so that the operator may physically “feel” the amount and rate of air being delivered to the recipient. Additional air pressure sensors and flow rate/volume sensors may be coupled in-line with the ventilator fluid flow path to assist with operating the device. The ventilator device’s inlet and/or outlet port may be sized for standard 22 mm fittings used for coupling to stock medical respiratory tubing. A multi-port endcap attachment may be coupled to the inlet port to allow for delivery of both air and supplemental oxygen.

For simplified operation, the ventilator device may be strapped to the operator’s leg (e.g., for ease of transport and operation), placed on a table (e.g., for single-hand operation), or held by the operator (e.g., for two-handed operation). Disclosed ventilator devices may be used with an intubation tube or a face mask. The air bellows may be shaped and sized to hold approximately 1.5 fluid liters and deliver about 500-700 milliliters per second (mls) per stroke of air, tank-fed oxygen, or a combination of both. The concertinaed bellows may fully open and close over a range of about 20-80 degrees or, in at least some configurations, about 55-65 degrees, e.g., to provide the best tactile feedback to the operator. Hand-operated handles may be provided and may be sized to fit a majority of adult hand sizes in the general population. The ventilator device may be equipped with a spring or other biasing member to passively open the device without reducing tactile feedback. Internal fluid channels are sized to move air over predefined feed volumes with minimal resistance.

To reduce operator fatigue, pumping of the ventilator may be achieved with shoulder, chest, back, leg, and/or arm muscles to help ensure the device can be comfortably operated for long periods of time. Disclosed designs may enable a lone person to ventilate a patient for a longer duration as compared to use of a handheld bag valve mask (BVM)-type ventilator. Clamshell, accordion-style designs enable an operator to employ physical leverage and, thus, reduce fatigue and extend time of operational use. Moreover, manual designs do not require electrical power, enabling assisted ventilation in settings that lack electric utility or generator supply such that electric ventilator machines cannot be used. In this regard, ventilator designs disclosed herein do not require sensors or electronics to operate, which reduces overall device cost and simplifies manufacturing. Most device parts may be mass produced from inexpensive materials using standard injection molding processes. In addition, the ventilator device employs fewer components and parts that are relatively simple to assemble, offering a concomitant reduction in molds and assembly time for manufacturing the device.

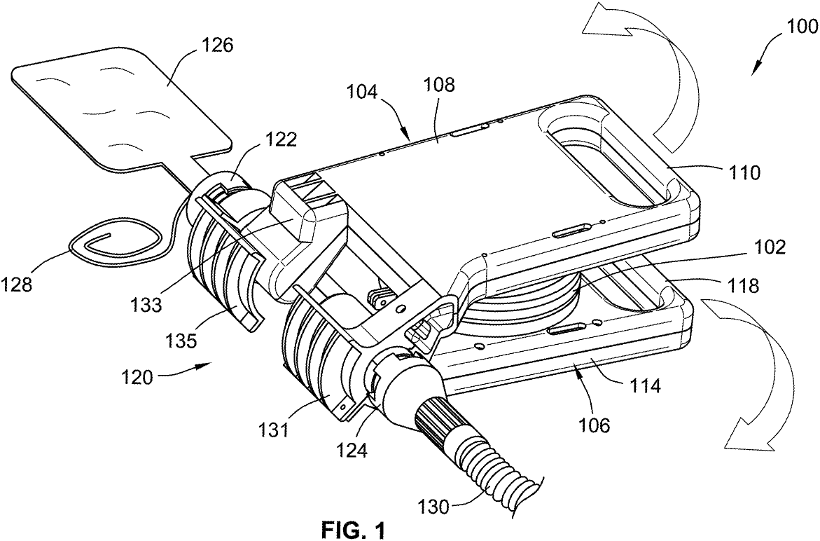

Referring now to the drawings, wherein like reference numbers refer to like features throughout the several views, FIG. 1 shows a representative manual ventilator, which is designated generally at 100 and portrayed herein for purposes of discussion as a single-use, hand-pumped portable ventilator device for providing assisted respiratory ventilation. The illustrated portable ventilator device 100 — also referred to herein as “ventilator device” or “ventilator” for short — is merely an exemplary application with which novel aspects of this disclosure may be practiced. In the same vein, incorporation of the present concepts for mask-based ventilation of a medical patient should also be appreciated as a non-limiting implementation of the disclosure. As such, it will be understood that aspects and features of this disclosure may be utilized for both intubation and mask-based applications, may be designed for private and professional use, and may be implemented for any logically relevant ventilator architecture. Moreover, only select components of the ventilator devices are shown and described in additional detail herein. Nevertheless, the ventilator devices discussed below may include numerous additional and alternative features, and other available peripheral components, for carrying out the various features and functions of this disclosure.

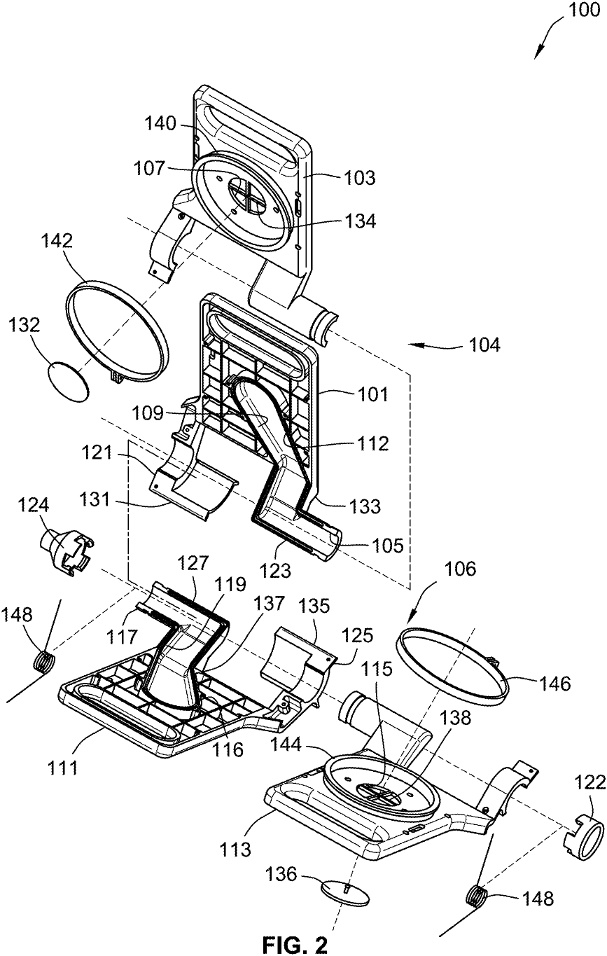

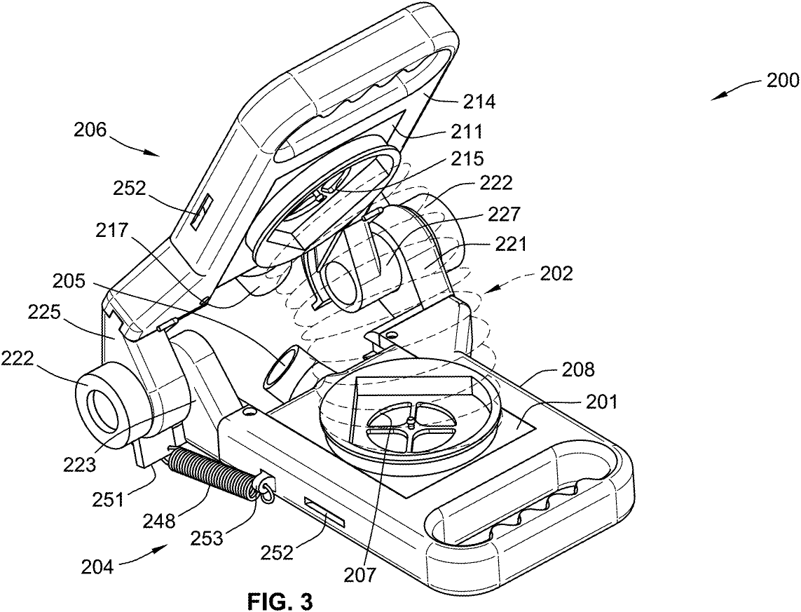

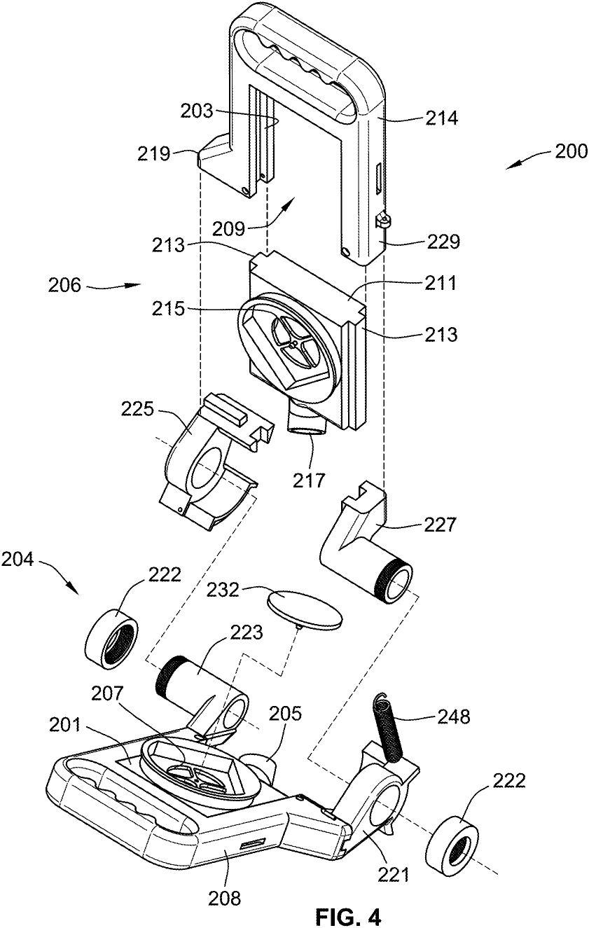

Ventilator device 100 of FIGS. 1 and 2 is portrayed with a clamshell, accordion-style form factor in which a pleated bellows 102 (shown compressed in FIG. 1) is interposed between and fluidly sealed to a pair of pivot-hinged panels 104 and 106 such that moving or “pumping” the panels 104, 106 towards and away from each other draws air/oxygen into and then expels air/oxygen out of the bellows 102. In accord with the illustrated example, an intake panel 104 — also referred to herein as “first panel” — is a bipartite construction with an elongated (first) panel body 108 (FIG. 1) composed of mating (first) panel halves 101 and 103 (FIG. 2). As opposing to a two-piece configuration, each panel body 108, 114 may be fabricated as a single-piece construction or with more than two components. As will be described below with reference to the ventilator device 200 of FIGS. 3 and 4, for example, a panel body may comprise four discrete components that are individually formed and subsequently assembled together.

When mated together, the panel halves 101, 103 of the intake panel body 108 collectively define an inlet port 105 through which air/oxygen is drawn into the ventilator 100 via the intake panel 104, and an outlet port 107 through which air/oxygen is expelled from the intake panel 104 and into the bellows 102. An internal channel 109 inside the panel body 108 fluidly connects the inlet port 105 to the outlet port 107. A polymeric gasket 112 is seated inside complementary cavities recessed into internal surfaces of the panel halves 101,103; the gasket 112 demarcates the outer perimeter of and seals the internal channel 109. As seen more clearly in FIG. 2, the intake panel’s inlet port 105 opens through a proximal (first) end of the panel body 108 (i.e., a longitudinal end closest to the pivot joint), whereas the intake panel’s outlet port 107 opens through an inward-facing (first) major face of the panel body 108. A distal end of the panel body 108 (i.e., a longitudinal end farthest from the pivot joint) is fabricated with a hand-operated (first) handle 110.

An exhaust panel 106 — also referred to herein as “second panel” — is juxtaposed with the intake panel 108 to sandwich therebetween the air bellows 102. For simplicity of design and ease of manufacture, the two panels 104, 106 may be substantially structurally identical to each other. For instance, the exhaust panel 106 may also be a bipartite construction with an elongated (second) panel body 114 (FIG. 1) composed of mating (second) panel halves 111 and 113 (FIG. 2). When mated together, these panel halves 111, 113 collectively define an inlet port 115 through which air/oxygen is pushed out from the bellows 102 and into the exhaust panel 106, and an outlet port 117 through which air/oxygen is expelled from the exhaust panel 106 and out of the ventilator 100. An internal channel 119 winds through the panel body 114 and fluidly connects the inlet port 115 to the outlet port 117. A polymeric gasket 116 extends around and seals the internal channel 119.

While structurally identical, the ventilator’s internal fluid-flow architecture is such that the exhaust panel’s inlet port 115 opens through an inward-facing (second) major face of the panel body 114, in opposing faced relation to the inlet panel’s outlet port 107 when the panels 104, 106 are pushed together and substantially parallel. Comparatively, the exhaust panel’s outlet port 117 opens through a longitudinal (second) end of the panel body 114, arranged coaxial with the inlet panel’s inlet port 105. An opposing longitudinal end of the panel body 114 is fabricated with a hand-operated (second) handle 118. It is envisioned that the ventilator device 100 may employ alternative handle designs or, optionally, may altogether omit the handles 110, 118.

With reference again to FIG. 1, a pivot hinge 120 pivotably attaches the distal end of the intake panel 104 to the distal end of the exhaust panel 106. While it is envisioned that the pivot hinge 120 may take on any suitable hinge construction, the hinge 120 is portrayed in FIG. 2 as a barrel-type hinge with a (first) barrel sleeve 121 and a (first) barrel cylinder 123 attached to the distal end of the intake panel 104, and a (second) barrel sleeve 125 and a (second) barrel cylinder 127 attached to the distal end of the exhaust panel 106. Internal channel 109 of the intake panel 104 originates at the outboard end of the barrel cylinder 123 with the inlet port 105, extends through the barrel cylinder 123 and panel body 108, and terminates at the outlet port 107. The outlet panel’s internal channel 119, on the other hand, originates at the major face of the panel body 114 with the inlet port 115, extends through the panel body 108, and terminates at the outboard end of the barrel cylinder 127 with the outlet port 107.

After individually assembling the two panels 104, 106, the inlet panel’s barrel cylinder 123 is slidably inserted into and rotates inside of the exhaust panel’s barrel sleeve 125. In the same vein, the exhaust panel’s barrel cylinder 127 is slidably inserted into and rotates inside of the inlet panel’s barrel sleeve 121. As shown, the inlet panel’s barrel sleeve 121 and barrel cylinder 123 are integrally formed with the inlet panel body’s two panel halves 101, 103, axially aligned on a common axis that projects transversely with respect to the panel body 108. In the same manner, the exhaust panel’s barrel sleeve 125 and barrel cylinder 127 are integrally formed with the exhaust panel body’s two panel halves 111, 113, axially aligned on the same common axis as the barrel sleeve 121 and barrel cylinder 123. Alternatively, the barrel cylinders and barrel sleeves may be fabricated as discrete components that are subsequently joined to their respective panel bodies and then mated together, as described below with respect to the ventilator device 200 of FIGS. 3 and 4.

Intake panel body 108 is formed with an arcuate (first) ribbed stopper 131 that is integrally formed with and projects axially from the barrel sleeve 121. Exhaust panel body 114 is also formed with an arcuate (second) ribbed stopper 135 that is integrally formed with and projects axially from its barrel sleeve 125. As the intake and exhaust panels 104, 106 are pivoted away from each other, the ribbed stopper 131 rotates in unison with the panel 104 and strikes a shoulder 137 (FIG. 2) that projects from the distal end of the exhaust panel’s elongated body 114 adjacent the barrel cylinder 127. The ribbed stopper 135 will concurrently rotate in unison with the panel 106 and strike a shoulder 133 projecting from the distal end of the panel body 108 adjacent the barrel cylinder 123. Doing so prevents the panel bodies 108, 114 from further angular movement and, thus, limits the total angle of rotation between the panels 104, 106 to about 55-65 degrees.

An annular inlet end cap 122 is snap-fit onto a complementary flange projecting radially outward from the distal tip of the barrel cylinder 123. Once secured in place, the inlet end cap 122 surrounds the inlet port 105 and mechanically attaches a reservoir bag 126 and an oxygen tube 128 to the intake panel 104, as best seen in FIG. 1. Inlet end cap 122 functions as an adapter lock for both fluidly coupling the reservoir bag 126 and oxygen tube 128 to the inlet port 105 and for allowing the bag 126 and tube 128 to swivel up to 360° with respect to the barrel cylinder 123.

A frustoconical outlet end cap 124 is snap-fit onto a complementary flange projecting radially outward from the distal tip of the barrel cylinder 127. Once secured in place, the outlet end cap 124 surrounds the outlet port 117 and mechanically attaches a patient air tube 130 (e.g., a single-use, 22 mm diameter corrugated ventilator tube) to the exhaust panel 106. Similar to inlet end cap 122, the outlet end cap 124 functions as an adapter lock for both fluidly coupling the air tube 130 to the outlet port 117 and for allowing the air tube 130 to swivel up to 360° with respect to the barrel cylinder 127.

Fluid flow into and out of the ventilator device 100 of FIGS. 1 and 2 may be governed by a distributed array of fluid-flow valves. For instance, a pair of one-way airflow valves are packaged in serial fluid flow communication between inlet port 105 and outlet port 117 to control the volume of air/oxygen drawn into the bellows 102 and the volume of air/oxygen pushed out of the bellows 102. An assortment of active and passive valve types may be employed in this manner, including flutter valves, ball valves, diaphragm valves, and other check valve configurations. Although described herein with two passively activated flapper valves, the ventilator device 100 may employ greater or fewer air flow valves of similar or distinct construction.

In accord with the illustrated example, a one-way (first) flapper valve, represented herein by a flexible flapper disk 132 and crosshair mounting interface 134, is fluidly connected to the intake panel’s outlet port 107. The crosshair mounting interface 134 is integrally formed with the bottom panel half 103 of the panel body 108 and extends across the outlet port 107. The center of the flapper disk 132 is mounted, e.g., via a fastener or a rivet, to the cruciform center of the crosshair mounting interface 134. This flapper valve functions to passively restrict airflow through the outlet port 107 in an outward (first) direction (i.e., from the bellows 102 back into the intake panel 104). In particular, as the panels 104, 106 are pressed together, fluid inside the bellows 102 is compressed; the now-pressurized fluid pushes on the flapper disk 132. This causes the flapper disk 132 to seat against the crosshair mounting interface 134 and exterior surface of the panel half 103 adjacent the port 107, sealing to the outer perimeter of the outlet port 107.

Another (second) one-way flapper valve, which is represented herein by a flexible flapper disk 136 and crosshair mounting interface 138, is fluidly connected to the exhaust panel’s inlet port 115. This crosshair mounting interface 138 is integrally formed with the top panel half 113 of the panel body 108 and extends across the inlet port 115. The center of the flexible flapper disk 136 is mounted, e.g., via a fastener or a rivet, to the cruciform center of the crosshair mounting interface 138. This flapper valve functions passively to restrict airflow through the inlet port 115 in an inward (second) direction (i.e., into the bellows 102 from the exhaust panel 106). In particular, as the panels 104, 106 are pulled apart, a vacuum pressure is created inside the bellows, which creates a pulling force on the flapper disk 136. This force of low pressure causes the flapper disk 136 to seat against the crosshair mounting interface 138 and the interior surface of the panel half 113 surrounding the port 115, sealing to the perimeter of the inlet port 115.

A fluid-tight bladder is placed in-line with the ventilator device’s inlet port 105 and outlet port 117 and functions to move fluid through the ventilator 100 during stroking or “pumping” of the intake and exhaust panels 104, 106. In a representative example, a concertinaed bellows 102, which is located between and physically attached to the panels 104, 106, fluidly connects to the intake panel’s outlet port 107 and the exhaust panel’s inlet port 115. Projecting downward in FIG. 2 from the major face of the bottom panel half 103 of panel body 108 is an annular (first) mounting interface 140 that surrounds the outlet port 107. Projecting upward in FIG. 2 from the major face of the top panel half 113 of panel body 114 is another annular (second) mounting interface 144 that surrounds the inlet port 115. Opposing top and bottom ends of the bellows 102 are slid onto the upper and lower mounting interfaces 140, 144, respectively, such that each end of the bellows 102 circumscribes and seals over a respective port 107, 115. The ends of the bellows 102 are then secured in place by hose clamps 142 and 146 such that the bellows 102 is physically coupled to the intake and exhaust panels 104, 106, respectively. Each mounting interface 140, 144 may in one embodiment have a cylindrical shape with an annual lip projecting radially outward at one end thereof.

By fluidly coupling the bellows 102 to the ports 107, 115 and mounting the bellows 102 to the panels 104, 106, pivoting the intake and exhaust panels 104, 106 away from each other causes the bellows 102 to expand and concomitantly create an internal low pressure or “vacuum” pressure. This vacuum pressure unseats the intake panel’s flapper disk 132, seats the exhaust panel’s flapper disk 136, and draws fluid into the bellows 102 through the intake panel 104 via inlet port 105. Conversely, pivoting the two panels towards each other causes the bellows 102 to contract (as shown in FIG. 1) and concomitantly create an internal fluid pressure. This fluid pressure seats the intake panel’s flapper disk 132, unseats the exhaust panel’s flapper disk 136, and pushes fluid out of the bellows 102 through the exhaust panel 106. An optional set of biasing members, such as torsion springs 148, is compressed or otherwise operatively connected between the panels 104, 106 and functions to bias or force apart the panels 104, 106, thereby minimizing the effort or work required to operate or “pump” the ventilator device 100.

Turning next to FIGS. 3 and 4, another representative manual ventilator 200 is shown for providing assisted respiratory ventilation. Although ventilator 100 and ventilator 200 differ in appearance, any of the features and options described above with reference to the ventilator device 100 of FIGS. 1 and 2 can be incorporated, singly or in any combination, into the ventilator device 200 of FIGS. 3 and 4, and vice versa. For example, ventilator device 200 is also portrayed with a clamshell, accordion-style form factor in which a pleated bellows 202 is interposed between and fluidly sealed to a pair of pivot-hinged panels 204 and 206 such that pumping the panels 204, 206 towards and away from each draws air/oxygen into and expels air/oxygen out from the bellows 202. As another point of similarity, the ventilator device 200 may employ the same flapper valves — flapper disks 132, 136 and crosshair mounting interfaces 134, 138 — and flapper valve arrangement described above with respect to FIGS. 1 and 2. FIG. 4, for example, shows the intake panel 204 with a flexible flapper disk 232 operatively mounted to seal and unseal outlet port 207 in a manner similar to how flexible flapper disk 132 selectively seals and unseals outlet port 107. Likewise, the bellows 202 of FIG. 3 may be structurally identical to the bellows 102. The intake and exhaust panels 204, 206 may also be structurally identical to each other. For purposes of brevity and efficiency, these overlapping features will not be described again in detail for the discussion of ventilator device 200.

Unlike the ventilator device 100 of FIGS. 1 and 2, however, the intake and exhaust panels 204, 206 of ventilator device 200 are each quadripartite constructions assembled from four mating parts: a U-shaped panel body 208, 214; an air module 201, 211; a barrel sleeve joint 221, 225; and a barrel cylinder joint 223, 227. In one instance, each air module 201, 211 is formed with guide rails 213 (FIG. 4) that project transversely from the module and extend continuously along the longitudinal length of the module. When the air module 211 is inserted into a central cavity 209 of the panel body 214, these guide rails 213 are received inside complementary guide slots 203 of the panel body 214. The air module 211 may then be secured in place, for example, via adhesives, heat stakes, screws, bolts, tapered surfaces, etc. The barrel sleeve joint 225 is then mounted to a left leg 229 of the panel body 214, whereas the barrel cylinder joint 227 is mounted to a right leg 221 of the panel body 214. Threaded end caps 222 lock the barrel cylinder joints 223, 227 within their respective barrel sleeve joint 221, 225 to pivotably hinge the intake panel 204 to the exhaust panel 206. Barrel sleeve joints 221, 225 may have an inner diameter that is sufficiently large to allow standardized tubing, such as patient air tube 130 of FIG. 1, to pass therethrough.

Air modules 201, 211 act as the ventilator’s fluid interface for moving air/oxygen between the tubing (e.g., oxygen tube 128 and patient air tube 130 of FIG. 1) and the bellows 202. As shown, the inlet port 205 of intake panel 204 projects longitudinally at an oblique angle from the air module 201, whereas the outlet port 217 of exhaust panel 206 projects longitudinally at an oblique angle from the air module 211. With this modular design, all air/oxygen flows into and out of the air modules 201, 211 via inlet ports 205, 215 and outlet ports 207, 217, rather than flowing through internal fluid conduits within the U-shaped panel bodies 208, 214. It may be desirable that each module 201, 211 be integrally formed as a single-piece, unitary structure that is fully enclosed except for the inlet/outlet ports that attach to tubing and the inlet/outlet ports that attach to the bellows. This modular design therefore eliminates the need for the gaskets 112, 116 of FIG. 2.

As another representative, non-limiting point of demarcation between the two illustrated manual ventilators, the ventilator device 200 of FIGS. 3 and 4 employs helical extension springs 248, in addition to or as an alternative for the torsional springs 148, for biasing the panels 204, 206 apart from each other. In this example, each barrel sleeve 221, 225 may include a barrel-side attachment flange 251 for coupling to one end of an extension springs 248, and each U-shaped panel body 208, 214 may include a body-side attachment flange 253 for coupling to the opposite end of the springs 248. When the ventilator 200 is closed, the panels 204, 206 are pushed together and the attachment flanges 251, 253 are concomitantly rotated away from each other. This movement causes the extension springs 248 to expand. Upon release of the panels 204, 206 by the operator, the spring force of the extension springs 248 will pull the panels 204, 206 apart. It may be desirable that only one type of spring configuration be used for a particular ventilator (e.g., ventilator 100 may employ only torsion springs 148, whereas ventilator 200 may employ only extension springs 248). Alternatively, the illustrated ventilators 100, 200 may employ any suitable type and combination of biasing members.

Both of the illustrated ventilators 100 and 200 are lightweight and portable devices for ease of use and transportation. The entire ventilator 200 of FIGS. 3 and 4, for example, may have a joint-to-handle height of approximately 11 inches and an end cap-to-cap width of approximately 10.5 inches offering a total footprint of less than one square foot. When the panels 204, 206 are pressed together such that the bellows 202 is in a fully compressed state, the ventilator 200 may have a panel-to-panel thickness of approximately 3.5 inches. When the panels 204, 206 are moved apart such that the bellows 202 is in a fully extended state, the intake panel 204 may be at about a 60 degree angle with respect to the exhaust panel 206. Optional strap slots 252 receive straps (not shown) to facilitate holding the ventilator device 200 or strapping the device 200 to a work surface or a person’s appendage.

Also presented herein are methods for manufacturing and methods for operating a human-powered ventilator device. In an example, a method is presented for assembling a ventilator device, such as manual ventilator devices 100 and 200 of FIGS. 1-4. The representative method includes receiving first and second panels, which may entail forming and assembling the constituent parts of the individual panels or merely collecting and arranging the previously assembled panels. As described above, each panel defines therein a respective inlet port, a respective outlet port, and a respective internal channel that fluidly connects the inlet and outlet ports. The method of assembly may then proceed to fluidly connect a one-way valve to the first panel’s outlet port; this one-way valve restricts airflow through the first outlet port in a first (inward) direction. Likewise, another one-way valve is fluidly connected to the second panel’s inlet port; this one-way valve restricts airflow through the second inlet port in a second (outward) direction opposite the first direction.

Prior to, contemporaneous with, or after connecting the one-way valves to their respective ports, the method includes pivotably attaching the first panel to the second panel, e.g., in a clamshell configuration. The method then fluidly connects a pleated and collapsible bellows to the first panel’s outlet port and the second panel’s inlet port. The bellows are also physically attached to the two panels such that the bellows is interposed between the panels. With this arrangement, pivoting the panels away from each other expands the bellows; in so doing, air is drawn into the ventilator through the first panel and into the bellows. Conversely, pivoting the panels towards each other compresses the bellows; doing so expels air from the bellows and out of the ventilator device through the second panel.

The method may also include attaching one or more biasing members to the pivotably attached panels. The biasing member(s) may be a pair of helical springs or torsion springs that bias the two panels away from each other. Fluidly connecting the bellows to the first panel’s outlet port and the second panel’s inlet port may include sliding the bellows onto annular mounting interfaces of the two panels such that the opposing ends of the bellows each surrounds a respective port. As yet a further option, attaching the bellows to the two panels may include rigidly mounting the bellows to the annular mounting interfaces, e.g., via clamps, straps, threaded fasteners, adhesives, etc.

Pivotably attaching the panels to each other may include pivotably attaching the longitudinal end of one panel to the longitudinal end of the other panel via a pivot hinge. The pivot hinge may be a barrel-type hinge with a respective barrel sleeve and a respective barrel cylinder attached to the longitudinal end of each panel. In this instance, pivotably attaching the panels to each other includes inserting the first panel’s barrel cylinder into the second panel’s barrel sleeve, and inserting the second panel’s barrel cylinder into the first panel’s barrel sleeve. Likewise, the one-way valves may be flapper-type valves with flexible flapper discs. In this instance, fluidly connecting the valves to the panels may include seating a first flapper disk against an exterior surface of the first panel and coupling the disk to the first panel’s body, seating a second flapper disk against an interior surface of the second panel and coupling the disk to the second panel’s body.

Additional features may be reflected in the following clauses:

Clause 1: a ventilator device, comprising: a first panel defining a first inlet port, a first outlet port, and a first internal channel fluidly connecting the first inlet port and the first outlet port; a first one-way valve fluidly connected to the first outlet port and restricting airflow therethrough in a first direction; a second panel pivotably attached to the first panel, the second panel defining a second inlet port, a second outlet port, and a second internal channel fluidly connecting the second inlet port and the second outlet port; a second one-way valve fluidly connected to the second inlet port and restricting airflow therethrough in a second direction opposite the first direction; and a bellows fluidly connected to the first outlet port and the second inlet port, the bellows interposed between and attached to the first and second panels such that pivoting the first and second panels away from each other expands the bellows and pivoting the first and second panels towards each other compresses the bellows.

Clause 2: the ventilator device of clause 1, wherein the first panel includes an elongated first panel body with a first longitudinal end defining therethrough the first inlet port and a first major face defining therethrough the first outlet port.

Clause 3: the ventilator device of clause 1 or clause 2, wherein the second panel includes an elongated second panel body with a second longitudinal end defining therethrough the second outlet port and a second major face defining therethrough the second inlet port.

Clause 4: the ventilator device of clause3, wherein the first panel includes a first annular mounting interface projecting from the first major face and surrounding the first outlet port, and the second panel includes a second annular mounting interface projecting from the second major face and surrounding the second inlet port, and wherein the bellows is mounted to the first and second annular mounting interfaces.

Clause 5: the ventilator device of any one of clauses 1 to 4, wherein the first panel and the second panel are substantially structurally identical.

Clause 6: the ventilator device of any one of clauses 1 to 5, further comprising a pivot hinge pivotably attaching a first end of the first panel to a second end of the second panel.

Clause 7: the ventilator device of clause 6, wherein the pivot hinge includes a first barrel sleeve and a first barrel cylinder attached to the first end of the first panel, and a second barrel sleeve and a second barrel cylinder attached to the second end of the second panel, and wherein the first barrel cylinder rotates inside the second barrel sleeve and the second barrel cylinder rotates inside the first barrel sleeve.

Clause 8: the ventilator device of clause 7, wherein the first barrel sleeve and the first barrel cylinder are integrally formed with the first panel, the second barrel sleeve and the second barrel cylinder are integrally formed with the second panel, the first internal channel extends through the first barrel cylinder, and the second internal channel extends through the second barrel cylinder.

Clause 9: the ventilator device of any one of clauses 1 to 8, wherein the first one-way valve includes a first flapper valve with a first flapper disk seated against an exterior surface of the first panel, and wherein the second one-way valve includes a second flapper valve with a second flapper disk seated against an interior surface of the second panel.

Clause 10: the ventilator device of any one of clauses 1 to 9, wherein the first panel includes a first hand-operated handle and the second panel includes a second hand-operated handle.

Clause 11: the ventilator device of any one of clauses 1 to 10, further comprising a biasing member biasing the first panel and the second panel away from each other.

Clause 12: the ventilator device of clause 11, wherein the biasing member includes a pair of helical springs or a pair of torsion springs.

Clause 13: a method of assembling a ventilator device, the method comprising: receiving a first panel defining a first inlet port, a first outlet port, and a first internal channel fluidly connecting the first inlet and outlet ports; fluidly connecting a first one-way valve to the first outlet port, the first one-way valve restricting airflow through the first outlet port in a first direction; pivotably attaching a second panel to the first panel in a clamshell configuration, the second panel defining a second inlet port, a second outlet port, and a second internal channel fluidly connecting the second inlet and outlet ports; fluidly connecting a second one-way valve to the second inlet port, the second one-way valve restricting airflow through the second inlet port in a second direction opposite the first direction; fluidly connecting a bellows to the first outlet port and the second inlet port; and attaching the bellows to the first and second panels with the bellows interposed between the first and second panels such that pivoting the first and second panels away from each other expands the bellows, drawing air through the first panel and into the bellows, and pivoting the first and second panels towards each other compresses the bellows, expelling air from the bellows and out through the second panel.

Clause 14: the method of clause 13, wherein the first panel includes an elongated first panel body with a first longitudinal end defining therethrough the first inlet port and a first major face defining therethrough the first outlet port, and the second panel includes an elongated second panel body with a second longitudinal end defining therethrough the second outlet port and a second major face defining therethrough the second inlet port.

Clause 15: the method of clause 14, wherein the first panel includes a first annular mounting interface projecting from the first major face and surrounding the first outlet port, and the second panel includes a second annular mounting interface projecting from the second major face and surrounding the second inlet port, and wherein attaching the bellows to the first and second panels includes rigidly mounting the bellows to the first and second annular mounting interfaces.

Clause 16: the method of any one of clauses 13 to 15, wherein pivotably attaching the second panel to the first panel includes pivotably attaching a first end of the first panel to a second end of the second panel via a pivot hinge.

Clause 17: the method of clause 16, wherein the pivot hinge includes a first barrel sleeve and a first barrel cylinder attached to the first end of the first panel, and a second barrel sleeve and a second barrel cylinder attached to the second end of the second panel, and wherein pivotably attaching the second panel to the first panel includes inserting the first barrel cylinder into the second barrel sleeve and inserting the second barrel cylinder into the first barrel sleeve.

Clause 18: the method of clause 17, wherein the first barrel sleeve and the first barrel cylinder are integrally formed with the first panel, the second barrel sleeve and the second barrel cylinder are integrally formed with the second panel, the first internal channel extends through the first barrel cylinder, and the second internal channel extends through the second barrel cylinder.

Clause 19: the method of any one of clauses 13 to 18, wherein the first one-way valve includes a first flapper valve with a first flapper disk seated against an exterior surface of the first panel, and wherein the second one-way valve includes a second flapper valve with a second flapper disk seated against an interior surface of the second panel.

Clause 20: the method of any one of clauses 13 to 19, further comprising attaching a biasing member to the first and second panels, the biasing member biasing the first and second panels away from each other.

While several modes for carrying out the many aspects of the present teachings have been described in detail, those familiar with the art to which these teachings relate will recognize various alternative aspects for practicing the present teachings that are within the scope of the appended claims. It is intended that all matter contained in the above description or shown in the accompanying drawings shall be interpreted as illustrative and exemplary of the range of possible embodiments that an ordinarily skilled artisan would recognize as implied by, structurally and/or functionally equivalent to, or otherwise rendered apparent based upon the included content, and not as limited solely to those explicitly depicted and/or described embodiments. Moreover, the present concepts expressly include combinations and sub-combinations of the described elements and features. The detailed description and the drawings are supportive and descriptive of the present teachings, with the scope of the present teachings defined solely by the claims.

What is claimed:

1. A ventilator device, comprising:
a first panel defining a first inlet port, a first outlet port, and a first internal channel fluidly connecting the first inlet port and the first outlet port;
a first one-way valve fluidly connected to the first outlet port and restricting airflow therethrough in a first direction;
a second panel pivotably attached to the first panel, the second panel defining a second inlet port, a second outlet port, and a second internal channel fluidly connecting the second inlet port and the second outlet port;
a second one-way valve fluidly connected to the second inlet port and restricting airflow therethrough in a second direction opposite the first direction; and
a bellows fluidly connected to the first outlet port and the second inlet port, the bellows interposed between and attached to the first and second panels such that pivoting the first and second panels away from each other expands the bellows and pivoting the first and second panels towards each other compresses the bellows.

2. The ventilator device of claim 1, wherein the first panel includes an elongated first panel body with a first longitudinal end defining therethrough the first inlet port and a first major face defining therethrough the first outlet port.

3. The ventilator device of claim 2, wherein the second panel includes an elongated second panel body with a second longitudinal end defining therethrough the second outlet port and a second major face defining therethrough the second inlet port.

4. The ventilator device of claim 3, wherein the first panel includes a first annular mounting interface projecting from the first major face and surrounding the first outlet port, and the second panel includes a second annular mounting interface projecting from the second major face and surrounding the second inlet port, and wherein the bellows is mounted to the first and second annular mounting interfaces.

5. The ventilator device of claim 1, wherein the first panel and the second panel are substantially structurally identical.

6. The ventilator device of claim 1, further comprising a pivot hinge pivotably attaching a first end of the first panel to a second end of the second panel.

7. The ventilator device of claim 6, wherein the pivot hinge includes a first barrel sleeve and a first barrel cylinder attached to the first end of the first panel, and a second barrel sleeve and a second barrel cylinder attached to the second end of the second panel, and wherein the first barrel cylinder rotates inside the second barrel sleeve and the second barrel cylinder rotates inside the first barrel sleeve.

8. The ventilator device of claim 7, wherein the first barrel sleeve and the first barrel cylinder are integrally formed with the first panel, the second barrel sleeve and the second barrel cylinder are integrally formed with the second panel, the first internal channel extends through the first barrel cylinder, and the second internal channel extends through the second barrel cylinder.

9. The ventilator device of claim 1, wherein the first one-way valve includes a first flapper valve with a first flapper disk seated against an exterior surface of the first panel, and wherein the second one-way valve includes a second flapper valve with a second flapper disk seated against an interior surface of the second panel.

10. The ventilator device of claim 1, wherein the first panel includes a first hand-operated handle and the second panel includes a second hand-operated handle.

11. The ventilator device of claim 1, further comprising a biasing member biasing the first panel and the second panel away from each other.

12. The ventilator device of claim 11, wherein the biasing member includes a pair of helical springs or a pair of torsion springs.

13. A method of assembling a ventilator device, the method comprising:
receiving a first panel defining a first inlet port, a first outlet port, and a first internal channel fluidly connecting the first inlet and outlet ports;
fluidly connecting a first one-way valve to the first outlet port, the first one-way valve restricting airflow through the first outlet port in a first direction;
pivotably attaching a second panel to the first panel in a clamshell configuration, the second panel defining a second inlet port, a second outlet port, and a second internal channel fluidly connecting the second inlet and outlet ports;
fluidly connecting a second one-way valve to the second inlet port, the second one-way valve restricting airflow through the second inlet port in a second direction opposite the first direction;
fluidly connecting a bellows to the first outlet port and the second inlet port; and
attaching the bellows to the first and second panels with the bellows interposed between the first and second panels such that pivoting the first and second panels away from each other expands the bellows, drawing air through the first panel and into the bellows, and pivoting the first and second panels towards each other compresses the bellows, expelling air from the bellows and out through the second panel.

14. The method of claim 13, wherein the first panel includes an elongated first panel body with a first longitudinal end defining therethrough the first inlet port and a first maj or face defining therethrough the first outlet port, and the second panel includes an elongated second panel body with a second longitudinal end defining therethrough the second outlet port and a second major face defining therethrough the second inlet port.

15. The method of claim 14, wherein the first panel includes a first annular mounting interface projecting from the first major face and surrounding the first outlet port, and the second panel includes a second annular mounting interface projecting from the second major face and surrounding the second inlet port, and wherein attaching the bellows to the first and second panels includes rigidly mounting the bellows to the first and second annular mounting interfaces.

16. The method of claim 13, wherein pivotably attaching the second panel to the first panel includes pivotably attaching a first end of the first panel to a second end of the second panel via a pivot hinge.

17. The method of claim 16, wherein the pivot hinge includes a first barrel sleeve and a first barrel cylinder attached to the first end of the first panel, and a second barrel sleeve and a second barrel cylinder attached to the second end of the second panel, and wherein pivotably attaching the second panel to the first panel includes inserting the first barrel cylinder into the second barrel sleeve and inserting the second barrel cylinder into the first barrel sleeve.

18. The method of claim 17, wherein the first barrel sleeve and the first barrel cylinder are integrally formed with the first panel, the second barrel sleeve and the second barrel cylinder are integrally formed with the second panel, the first internal channel extends through the first barrel cylinder, and the second internal channel extends through the second barrel cylinder.

19. The method of claim 13, wherein the first one-way valve includes a first flapper valve with a first flapper disk seated against an exterior surface of the first panel, and wherein the second one-way valve includes a second flapper valve with a second flapper disk seated against an interior surface of the second panel.

20. The method of claim 13, further comprising attaching a biasing member to the first and second panels, the biasing member biasing the first and second panels away from each other.
